(19) United States
(12) Patent Application Publication
(10) Pub. No.: US 2022/0096051 A1
(43) Pub. Date: Mar. 31, 2022

(54) SCANNING ASSEMBLY FOR ULTRASONIC IMAGING DEVICE AND ULTRASONIC IMAGING DEVICE

(71) Applicant: GE Precision Healthcare LLC, Wauwatosa, WI (US)

(72) Inventors: Bing Li, Wuxi (CN); Qiang Yao, Wuxi (CN); Hongyu Zhao, Wuxi (CN); Lu Jin, Wuxi (CN); Liping Chen, Wuxi (CN); Kejian Shi, Wuxi (CN)

(21) Appl. No.: 17/490,618

(22) Filed: Sep. 30, 2021

(30) Foreign Application Priority Data
Sep. 30, 2020 (CN) 202011060155.6

(51) Int. Cl. A61B 8/00

(52) U.S. Cl. CPC A61B 8/4461; A61B 8/0825; A61B 8/46; A61B 8/4218

(57) ABSTRACT

A scanning assembly for an ultrasonic imaging device and the ultrasonic imaging device are provided in the present disclosure. The scanning assembly comprises: an ultrasonic transducer configured to send/receive an ultrasonic signal; a frame connected to an ultrasonic imaging device body; a lower housing comprising a hollow cavity having an upper open end and a lower open end, wherein the upper open end is detachably connected to the bottom of the frame, and the ultrasonic transducer is at least partially accommodated in the hollow cavity; and a film assembly detachably connected to the lower open end. The ultrasonic imaging device comprises the ultrasonic imaging device body. The ultrasonic imaging device body comprises a main device, a display, an adjustable arm, and the above scanning assembly connected to one end of the adjustable arm.

### CROSS-REFERENCE TO RELATED APPLICATIONS

This application claims priority to Chinese Patent Application No. 202011060155.6, filed on Sep. 30, 2020, the disclosure of which is incorporated herein by reference in its entirety.

### BACKGROUND

The present disclosure relates to the field of ultrasonic imaging, and in particular, relates to a scanning assembly for an ultrasonic imaging device and the ultrasonic imaging device.

An ultrasonic imaging device usually uses a scanning assembly including an ultrasonic transducer to emit an ultrasonic signal and receive an echo signal for imaging. Ultrasonic imaging devices can be used to scan a variety of human organs and tissues. For example, a full-field breast ultrasonic imaging device is one of them, and can perform ultrasonic imaging on the breast of a person to be scanned.

In some examples, the full-field breast ultrasonic imaging device includes an ultrasonic transducer partially accommodated in a housing and a film assembly having a film or film sheet. When a scan is performed, the above film assembly can be used to press the breast, and the ultrasonic transducer is in contact with the other side of the film or film sheet, and is translated over the film or film sheet to scan the breast. In order to meet hygienic requirements, the ultrasonic transducer usually needs to be cleaned after a certain number of scans have been performed. However, most ultrasonic transducers are accommodated in the housing, and therefore, execution of cleaning and sterilization operations is very inconvenient.

### SUMMARY

In one aspect of the present disclosure is a scanning assembly for an ultrasonic imaging device, comprising: an ultrasonic transducer configured to send/receive an ultrasonic signal; a frame connected to an ultrasonic imaging device body; a lower housing comprising a hollow cavity having an upper open end and a lower open end, wherein the upper open end is detachably connected to the bottom of the frame, and the ultrasonic transducer is at least partially accommodated in the hollow cavity; and a film assembly detachably connected to the lower open end.

In another aspect, the upper open end of the lower housing and the bottom of the frame are respectively provided with a plurality of guideposts and a plurality of guide holes corresponding to one another.

In another aspect, the upper open end of the lower housing and the bottom of the frame are respectively provided with a locking element and a mating element corresponding to the locking element.

In another aspect, the locking element comprises a pin, and the mating element comprises a pin hole.

In another aspect, the locking element comprises a hook, and the mating element comprises a recess.

In another aspect, the lower housing is made of a transparent material.

In another aspect, an upper housing is further included, and is detachably connected to the top of the frame.

In another aspect, the upper housing is made of a transparent material.

In another aspect, an illumination device is further included. The illumination device is arranged inside the scanning assembly, and is configured to illuminate tissue to be imaged in an ultrasonic imaging process.

In another aspect, a driving device is further included. The driving device is at least partially arranged in the frame, and is connected to the ultrasonic transducer to drive the ultrasonic transducer to move.

In another aspect, the driving device comprises a motor and a screw rod. The screw rod is arranged in the frame, and the motor is fixedly connected to the ultrasonic transducer and movably connected to the screw rod to drive the ultrasonic transducer to move.

In another aspect, the driving device further comprises a guide rail and a sliding block. The guide rail is fixedly arranged in the frame, and the sliding block is fixedly connected to the motor. The guide rail is in slide connection with the sliding block and is configured to perform a guiding function when the ultrasonic transducer moves.

In another aspect, the film assembly comprises an outer frame and a film. The film is fixedly arranged in the outer frame, and the outer frame is detachably connected to the lower open end of the lower housing. In an ultrasonic imaging process of the ultrasonic imaging device, one surface of the film is at least partially in contact with the ultrasonic transducer, and the other surface of the film is at least partially in contact with the tissue to be scanned.

In another aspect, the lower open end of the lower housing is provided with a first magnetic material, and the outer frame is provided with a second magnetic material correspondingly. The first magnetic material and the second magnetic materials attract each other through a magnetic force, such that the film assembly is connected to the lower open end of the lower housing.

In another aspect, a detection circuit is further included. The detection circuit comprises a magnetic induction switch arranged at the lower open end of the lower housing and an electrical connection assembly connecting the magnetic induction switch to a controller. When the film assembly is connected to the lower open end of the lower housing, the magnetic induction switch senses a magnetic field from the second magnetic material, and sends a signal to the controller through the electrical connection assembly.

In a further aspect of the present disclosure is an ultrasonic imaging device, comprising: an ultrasonic imaging device body comprising a main device, a display, and an adjustable arm; and the scanning assembly according to any one of the above descriptions, wherein the scanning assembly is connected to one end of the adjustable arm.

It should be understood that the summary above is provided to introduce, in a simplified form, some concepts that will be further described in the detailed description. This summary is not meant to identify key or essential features of the claimed subject matter. The protection scope is defined uniquely by the claims that follow the detailed description. Furthermore, the claimed subject matter is not limited to implementations that solve any disadvantages noted above or in any section of the present disclosure.

### BRIEF DESCRIPTION OF THE DRAWINGS

The present disclosure will be better understood by reading the following description of non-limiting embodiments with reference to the accompanying drawings.

FIG. 1 shows a perspective view of an ultrasonic imaging device according to an embodiment of the present disclosure;

FIG. 2 shows a schematic block diagram of various system components according to an embodiment of the present disclosure;

FIG. 3 shows a perspective view of the scanning assembly at one viewing angle according to an embodiment of the present disclosure;

FIG. 4 shows a three-dimensional exploded view of the scanning assembly at another viewing angle according to an embodiment of the present disclosure;

FIG. 5 shows a perspective view of the scanning assembly with an upper housing removed according to an embodiment of the present disclosure;

FIG. 6 is a schematic diagram showing a connection relationship between a frame and an ultrasonic transducer according to an embodiment of the present disclosure;

FIG. 7 shows a perspective view of a lower housing and a film assembly in an assembled state according to an embodiment of the present disclosure; and

FIG. 8 shows a perspective view of the film assembly at one angle according to an embodiment of the present disclosure.

### DETAILED DESCRIPTION

Specific aspects, features and embodiments of the present disclosure will be described. It should be noted that during the specific description of the implementations, it is impossible to describe all features of the actual implementations in detail in this description for the sake of briefness. It should be understood that in the actual implementation of any of the implementations, as in the process of any engineering project or design project, a variety of specific decisions are often made in order to achieve the developer's specific objectives and meet system-related or business-related restrictions, which will vary from one implementation to another. Moreover, it can also be understood that although the efforts made in such development process may be complex and lengthy, for those of ordinary skill in the art related to content disclosed in the present disclosure, some changes in design, manufacturing, production or the like based on the technical content disclosed in the present disclosure are only conventional technical features, and should not be construed as that the content of the present disclosure is insufficient.

Unless otherwise defined, the technical or scientific terms used in the claims and the description are as they are usually understood by those of ordinary skill in the art to which the present disclosure pertains. Terms such as “first,” “second,” and similar words used in this specification and claims do not denote any order, quantity, or importance, but are only intended to distinguish different constituents. “One,” “a(n),” and similar terms are not meant to be limiting, but rather denote the presence of at least one. The term “include,” “comprise,” or a similar term is intended to mean that an element or article that appears before “include” or “comprise” encompasses an element or article and equivalent elements that are listed after “include” or “comprise,” and does not exclude other elements or articles. The term “connect,” “connected,” or a similar term is not limited to a physical or mechanical connection, and is not limited to a direct or indirect connection.

Although some embodiments of the present disclosure are presented in the particular context of human breast ultrasound, it should be understood that the present disclosure is applicable to ultrasound scanning of any externally accessible human or animal body part (for example, abdomen, legs, feet, arms, or neck), and is also applicable to other medical imaging devices (for example, X-ray scanning) with a similar mechanical structure.

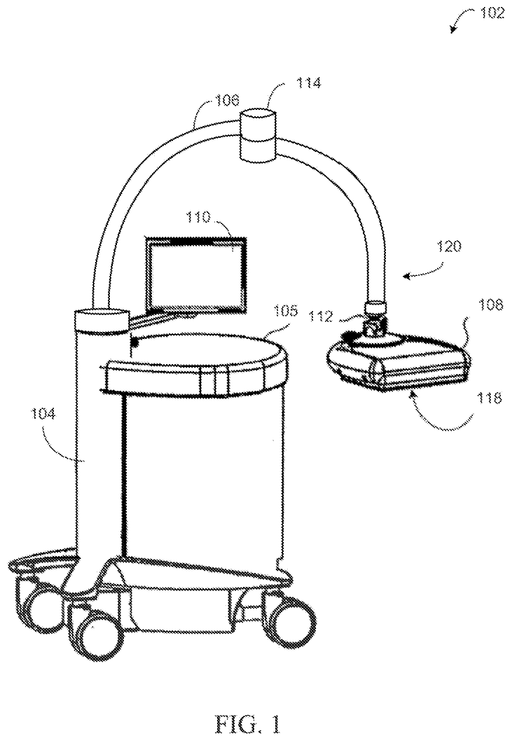

FIG. 1 shows a perspective view of an ultrasonic imaging device 102 (hereinafter also generally referred to as an imaging device 102) according to some embodiments. A body of the ultrasonic imaging device 102 may be a main device, a display 110, an adjustable arm 106, and a scanning assembly 108. The main device may include a body frame 104, an ultrasonic processor housing 105, and an ultrasonic processor inside the housing 105. The specific structure of each component will be illustrated in detail below.

The body frame 104, the ultrasonic processor housing 105 containing the ultrasonic processor, a movable and adjustable support arm (for example, an adjustable arm) 106 including a hinge joint 114, the scanning assembly 108 connected to a first end 120 of the adjustable arm 106 by means of a ball and socket connector (for example, a ball joint) 112, and the display 110 connected to the body frame 104. The display 110 is connected to the body frame 104 at a joining point where the adjustable arm 106 enters the body frame 104. Since the display 110 is directly connected to the body frame 104 rather than the adjustable arm 106, the display 110 does not affect the weight of the adjustable arm 106 and a balancing mechanism of the adjustable arm 106. In one example, the display 110 can rotate in horizontal and transverse directions (for example, rotatable around a central axis of the body frame 104), but cannot move vertically. In an alternative example, the display 110 may also be vertically movable. Although FIG. 1 illustrates the display 110 connected to the body frame 104, in other examples, the display 110 may be connected to different components of the imaging device 102, such as connected to the ultrasonic processor housing 105, or positioned away from the imaging device 102.

In one embodiment, the adjustable arm 106 is configured and adapted such that the pressing/scanning assembly 108 (i) is neutrally buoyant in space, or (ii) has a light downward net weight (for example, 1-2 kg) for pressing the breast, while allowing easy user operation. In an alternative embodiment, the adjustable arm 106 is configured such that the scanning assembly 108 is neutrally buoyant in space during positioning of a scanner on tissues of a patient. Then, after the scanning assembly 108 is positioned, internal components of the imaging device 102 may be adjusted to apply a desired downward weight to press the breast and improve image quality. In one example, the downward weight (for example, a force) may be in a range of 2-11 kg.

As described above, the adjustable arm 106 includes the hinge joint 114. The hinge joint 114 divides the adjustable arm 106 into a first arm portion and a second arm portion. The first arm portion is connected to the scanning assembly 108 and the second arm portion is connected to the body frame 104. The hinge joint 114 allows the second arm portion to rotate relative to the second arm portion and the body frame 104. For example, the hinge joint 114 allows the scanning assembly 108 to translate transversely and horizontally relative to the second arm portion and the body frame 104, but not vertically. In such manner, the scanning assembly 108 can rotate toward the body frame 104 or away from the body frame 104. However, the hinge joint 114 is configured to allow the entire adjustable arm 106 (for example, the first arm portion and the second arm portion) to move vertically together as a whole (for example, translating upward and downward along with the body frame 104).

The scanning assembly 108 may include a film assembly 118 having a film that is in a substantially tensioned state to be at least partially attached, and used to press the breast. The film assembly 118 has a bottom surface for contacting the breast, and when the bottom surface is in contact with the breast, the transducer sweeps over a top surface of the film assembly to scan the breast. In one example, the film is a tensioned fabric sheet.

In an exemplary embodiment, the adjustable arm may include a potentiometer (not shown) to sense the position and direction of the pressing/scanning assembly 108, or other types of position and direction sensing (such as gyroscope, magnetic, optical, and radio frequency (RF)) may be used. A full-function ultrasonic engine may be provided within the ultrasonic processor housing 105, and is configured to drive the ultrasonic transducer, and generate volumetric breast ultrasound data from a scan in conjunction with related position and orientation information. In some examples, volumetric scan data may be transmitted to another computer system by using any of a variety of data transmission methods known in the art so as to be further processed, or the volumetric scan data may be processed by the ultrasonic engine. A general-purpose computer/processor integrated with the ultrasonic engine may further be provided for general user interface and system control. The general-purpose computer may be a self-contained stand-alone unit, or may be remotely controlled, configured, and/or monitored by remote stations connected across networks.

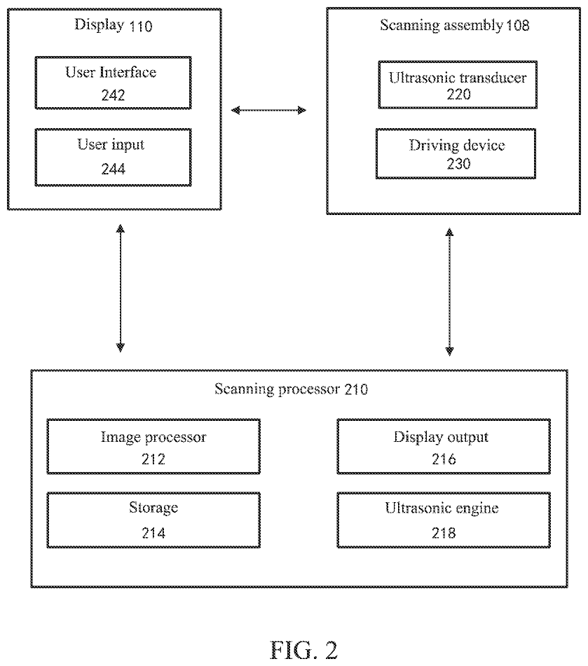

FIG. 2 is a block diagram 200 schematically showing various system components of the imaging device 102, including the scanning assembly 108, the display 110, and a scanning processor 210. In one example, the scanning processor 210 may be included in the ultrasonic processor housing 105 of the imaging device 102. As shown in the embodiment of FIG. 2, the scanning assembly 108, the display 110, and the scanning processor 210 are independent components that communicate with each other; however, in an exemplary embodiment, one or more of these components may be integrated (for example, the display and the scanning processor may be included in a single component).

First, refer to the scanning assembly 108, which at least includes an ultrasonic transducer 220 and a driving device 230. The ultrasonic transducer 220 includes a transducer array of transducer elements, such as a piezoelectric element that converts electrical energy into ultrasonic waves and then detects reflected ultrasonic waves. The structure of the driving device 230 will be described in detail below.

The scanning assembly 108 may communicate with the scanning processor 210 to send original scan data to an image processor. The scanning assembly 108 may optionally communicate with the display 110 so as to instruct a user to reposition the scanning assembly as described above, or to receive information from the user (via user input 244).

Now turn to the scanning processor 210, which includes an image processor 212, a memory 214, display output 216, and an ultrasonic engine 218. The ultrasonic engine 218 may drive activation of the transducer elements of the transducer 220, and in an exemplary embodiment, may activate the driving device 230. Furthermore, the ultrasonic engine 218 may receive original image data (e.g., ultrasonic echoes) from the scanning assembly 108. The original image data may be sent to the image processor 212 and/or a remote processor (e.g., via a network), and processed to form a displayable image of a tissue sample. It should be understood that in an exemplary embodiment, the image processor 212 may be included in the ultrasonic engine 218.

Information may be transmitted from the ultrasonic engine 218 and/or the image processor 212 to a user of the imaging device 102 via the display output 216 of the scanning processor 210. In an example, the user of the scanning device may be an ultrasonic technician, a nurse, or a physician such as a radiologist. For example, a processed image of scanned tissue may be sent to the display 110 via the display output 216. In another example, information related to scan parameters (such as a scan progress) may be sent to the display 110 via the display output 216. The display 110 may include a user interface 242 configured to display images or other information to the user. Furthermore, the user interface 242 may be configured to receive input from the user (such as by means of the user input 244), and send the input to the scanning processor 210. In one example, the user input 244 may be a touch screen of the display 110. However, other types of user input mechanisms are also possible, such as a mouse, a keyboard, and the like.

The scanning processor 210 may further include the memory 214. The storage 214 may include movable and/or permanent devices, and may include an optical memory, a semiconductor memory, and/or a magnetic memory. The storage 214 may include a volatile, non-volatile, dynamic, static, read/write, read only, random access, sequential access, and/or additional memory. The storage 214 may store non-transitory instructions executable by a controller or processor (such as the controller 218 or the image processor 212) so as to implement one or more methods or routines as described below. The storage 214 may store original image data received from the scanning assembly 108, processed image data received from the image processor 212 or a remote processor, and/or additional information.

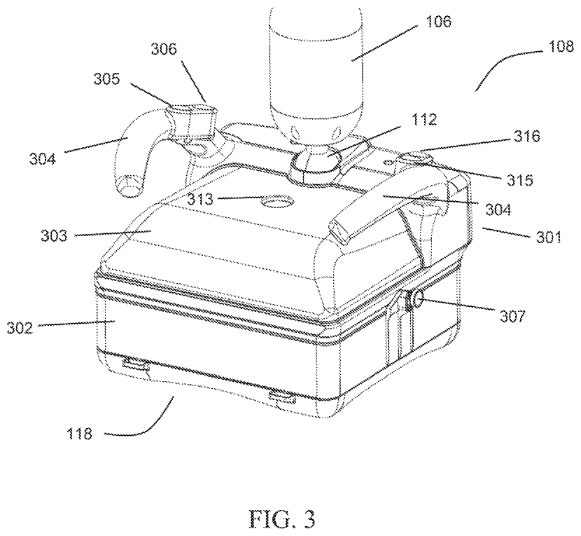

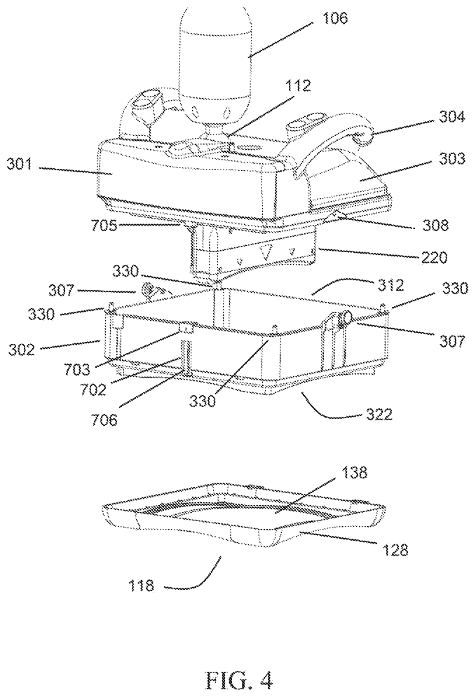

Continue to refer to FIG. 3 and FIG. 4. FIG. 3 shows a perspective view of the scanning assembly 108 at one viewing angle according to some embodiments of the present disclosure. FIG. 4 shows a three-dimensional exploded view of the scanning assembly 108 at another viewing angle. The scanning assembly 108 may include the ultrasonic transducer 220, a frame 301, a lower housing 302, and the film assembly 118. The ultrasonic transducer 220 is configured to send and receive ultrasonic signals. The frame 301 can be connected to an ultrasonic imaging device body, and a specific connection configuration will be described in detail below. The lower housing 302 may be a hollow cavity structure including an upper open end 312 and a lower open end 322. The shape of the hollow cavity may be configured to match the bottom of the frame 301 and the film assembly 118 respectively. In such manner, the upper open end 312 of the hollow cavity may be detachably connected to the bottom of the frame 301, and the lower open end 322 may be detachably connected to the film assembly 118. The hollow cavity of the lower housing 302 is configured to at least partially accommodate the ultrasonic transducer 220.

The scanning assembly 108 further includes two handles 304 arranged at the frame 301. The two handles 304 oppose each other across a transverse axis of the scanning assembly 108, and the transverse axis is centered on the adjustable arm 106 and defined relative to the transverse axis. The frame 301 may have a rectangular opening. In another example, the frame 301 may have another shape, such as a square having a square opening. In addition, the frame 301 has a thickness defined between an inner periphery and an outer periphery of the frame 301.

The two handles 304 are configured to move the scanning assembly 108 in space and to position the scanning assembly 108 on tissue (e.g., on a patient). In an alternative embodiment, the scanning assembly 108 may not include the handles 304. In an example, the handles 304 may be formed integrally with the frame 301. In another example, the handles 304 and the frame 301 may be formed separately.

As shown in FIG. 3, the scanning assembly 108 is connected to the adjustable arm 106 by means of the ball joint 112 (e.g., a ball and socket connector). Specifically, a top dome portion of the frame 301 is connected to the ball joint 112. The top of the frame 301 includes a depression forming a socket, and a ball of the ball joint 112 is fit in the socket. The ball joint 112 is movable in multiple directions. For example, the ball joint 112 enables rotational motion of the scanning assembly relative to the adjustable arm 106. The ball joint 112 includes a locking mechanism for locking the ball joint 112 in place, thereby holding the scanning assembly 108 stationary relative to the adjustable arm 106. Furthermore, the ball joint 112 may also be configured to only rotate but not to move in multiple directions, such as oscillating.

In addition, as shown in FIG. 3, the handles 304 are further provided with buttons for controlling scanning and adjusting the scanning assembly 108. Specifically, the buttons may include a first weight adjustment button 305 and a second weight adjustment button 306. The first weight adjustment button 305 may reduce a load applied to the scanning assembly 108 from the adjustable arm 106. The second weight adjustment button 306 may increase a load applied to the scanning assembly 108 from the adjustable arm 106. Increasing the load applied to the scanning assembly 108 may increase the pressure and the amount of pressing applied to the tissue on which the scanning assembly 108 is placed. Furthermore, increasing the load applied to the scanning assembly increases the effective weight of the scanning assembly on the tissue to be scanned. In one example, increasing the load may press tissue of a patient, such as that of the breast. In such way, varying amounts of pressure (e.g., load) may be applied consistently with the scanning assembly 108 during scanning in order to obtain high quality images by using the transducer module 220.

Prior to the scanning process, a user (e.g., an ultrasonic technician or physician) may position the scanning assembly 108 on a patient or tissue. Once the scanning assembly 108 is properly positioned, the user may adjust the pressure (e.g., adjusting an amount of pressing) of the scanning assembly 108 on the patient by using the first weight adjustment button 305 and/or the second weight adjustment button 306. The user may then initiate a scanning process by means of an additional control on the handles 304. For example, as shown in FIG. 3, the second handle of the handles 304 includes two additional buttons 315 and 316. The two additional buttons may include a first button for initiating a scan (e.g., once the scanning assembly has been placed on the tissue/patient and an amount of pressing has been selected) and a second button for stopping the scan. In one example, once the first button is selected, the ball joint 112 may be locked, thereby stopping movement of the scanning assembly 108, and starting the scanning of the ultrasonic transducer.

The scanning assembly 108 is configured to remain stationary during scanning. In other words, once the weight applied to the scanning assembly 108 is adjusted by means of the adjustable arm 106 and the ball joint 112 is locked, the scanning assembly 108 may remain in a resting position without translating in the horizontal or transverse direction. However, the scanning assembly 108 may still translate vertically along with the vertical movement of the adjustable arm 106.

The film assembly 118 may further include an outer frame 128 and a film 138. In an ultrasonic imaging process performed by the ultrasonic imaging device, one surface of the film 138 can be at least partially in contact with the ultrasonic transducer 220, and the other surface of the film 138 is at least partially in contact with tissue to be scanned. Such an arrangement can ensure that the ultrasonic transducer transmits and receives signals with less attenuation, and can fix the breast to be scanned to facilitate scanning.

In an assembled state shown in FIG. 3, the ultrasonic transducer 220 (not shown in FIG. 3) is completely accommodated in a combination of the frame 301, the lower housing 302, and the film assembly 118. When the user carries out ultrasonic scanning, mechanical movement of the ultrasonic transducer 220 will not cause harm to the body of a person being scanned. At the same time, the structure of the above combination can also protect the relatively expensive ultrasonic transducer 220.

The three-dimensional exploded view shown in FIG. 4 shows in detail positional relationships between various components when the lower housing 302 and the film assembly 118 of the present disclosure are in an unassembled state. The lower housing 302 of the present disclosure is detachably connected to the frame 301 and the film assembly 118, respectively. Compared with the direct connection between a frame 301 and a film assembly 118 in the prior art, the connection configuration of the present disclosure allows partial accommodation of the ultrasonic transducer 220 in the lower housing 302. In this way, after the lower housing 302 has been removed as shown in FIG. 4, most portions of the ultrasonic transducer 220, especially portions in direct or indirect contact with a scanned part of the person being scanned in the ultrasonic scanning process, can be completely exposed. When scans have been performed a certain number of times or other conditions are met and sterilization is required, an operator can easily clean and sterilize the ultrasonic transducer 220 and other internal structures of the scanning assembly 108. This not only saves time, but also enables more thorough sterilization and cleaning, thus improving the overall safety of using the device.

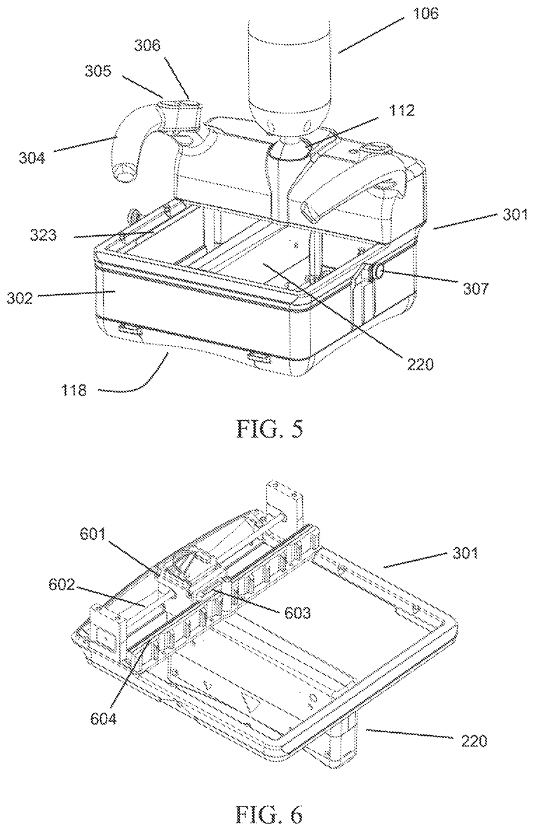

In an exemplary embodiment, the present disclosure may also include a guiding structure. Specifically, in order to realize quick assembly of the lower housing 302 and the frame 301 by a user and firm connection between the lower housing 302 and the frame 301 after the assembly, the upper open end 312 of the lower housing 302 and the bottom of the frame 301 may be respectively provided with a plurality of guideposts and a plurality of guide holes corresponding to one another. In an exemplary embodiment, the structure of the guidepost may be configured as shown in FIG. 4 and FIG. 5, that is, the guidepost 330 is arranged at the upper open end 312 of the lower housing 302. For example, four guideposts 330 are respectively arranged at four top corners of the upper open end 312. Correspondingly, guide holes (not shown) matching these guideposts are arranged at the bottom of the frame 301 corresponding to the positions of the guideposts 330. The size of the guide holes matches the size of the guideposts. When assembling the lower housing 302 and the frame 301, the user only needs to align the guide holes with the guideposts, thus completing the assembling easily. In addition, after the assembly is completed, the structural cooperation of the guide holes and the guideposts can also limit the positions of the frame 301 and the lower housing 302, thereby further improving the reliability of the assembly, and avoiding sliding of the frame 301 and the lower housing 302 relative to each other. In an exemplary embodiment, the guidepost 330 may be further configured as a structure with an end portion having a smaller outer diameter. For example, the end portion is configured as a tapered structure or a structure having an arc-shaped surface so as to further enhance the simplicity of assembly.

It should be noted that the example shown above is only a representative example. The positions at which the guideposts and the guide holes are arranged may be arbitrary. For example, the guideposts may be arranged at the bottom of the frame 301, and the guide holes may be arranged at the upper open end of the lower housing 302. In addition, the guideposts and the guide holes may be arranged at the top corners as described above, and may also be arranged at other positions, and the quantities thereof may also be freely selected.

In addition to the above guiding structure, in order to achieve a stable connection between the lower housing 302 and the frame 301, some embodiments of the present disclosure may also include a locking element and a mating element corresponding to the locking element which are respectively arranged at the frame 301 and the lower housing 302. Specifically, the locking element and the mating element may be arranged at the upper open end 312 of the lower housing 302 and the bottom of the frame 301, respectively. FIG. 4 shows the arrangement of the locking element and the mating element according to some embodiments of the present disclosure. The locking element may include a pin 307, and correspondingly, the mating element corresponding to the locking element may include a pin hole 308. The pin 307 may be movably connected to the lower housing 302 by means of a base arranged at the upper open end 312. When the lower housing 302 is assembled at a bottom end of the lower housing 301, the free movement direction of the pin 307 can be aligned with the pin hole 308. At this time, the pin 307 can at least partially enter the pin hole 308 when pressed, thereby achieving an engagement between the two. When disassembly is required, only the pin 307 needs to be pulled out. The quantity and position of the pin 308 may be arbitrary. For example, as shown in FIG. 4, two pins 308 are respectively arranged at two opposite edges of the lower housing 302. At this time, the position and quantity of the pin hole 308 should be configured accordingly.

It should be noted that the locking element and the mating element may also be configured in another manner, for example, in the form of a padlock. The locking element may be configured as a hook structure, such as a plastic elastic hook, and the mating element may include a recess structure matching the hook structure. Cooperation of the hook structure and the recess implements a detachable connection between the lower housing 302 and the frame 301.

In an exemplary embodiment, the scanning assembly 108 may further include an upper housing structure. For example, as shown in FIG. 3 and FIG. 4, the upper housing 303 is detachably connected to the top of the frame 301, and is located at the other side opposite to the lower housing 302. The upper housing 303 can cover the ultrasonic transducer 220 from the top, thus further protecting the user and the ultrasonic transducer 220. The ultrasonic transducer 220 will generate an airflow in the scanning assembly 108 in a moving process, and the upper housing 303 of the present disclosure may include an opening 313 for air circulation so as to prevent movement of the ultrasonic transducer 220 from being hindered. In addition, the existence of the opening 313 can also drain moisture inside the upper housing 303 promptly, thus preventing formation of water mist from obstructing interior observation performed by the user.

In an exemplary embodiment, the upper housing 303 may be configured to be detachably connected to the top of the frame 301. The detachable connection may be a mechanical connection, a magnetic connection, etc., which will not be described here. Referring to FIG. 5, a perspective view of the scanning assembly 108 with the upper housing 303 removed is shown. When the upper housing 303 is removed, internal components of the frame 301 can be seen from the top thereof. For example, the position of the ultrasonic transducer 220 can be clearly seen. In an exemplary embodiment, the interior of the frame 301 may further include a plurality of illumination devices 323. The illumination device 323 shown in FIG. 5 may be a strip-shaped LED lamp structure located on an inner wall of the frame 301. The illumination device 323 is used to illuminate the tissue to be imaged in the ultrasonic imaging process, so as to facilitate observation and recording operations performed by the user. It should be noted that the shape, position, and quantity of the illumination device 323 may also be of other types, which will not be described here.

In an exemplary embodiment, the upper housing 303 and the lower housing 302 may be made of a rigid transparent material. For example, rigid plastics such as polypropylene (PP), polymethyl methacrylate (PMMA), polystyrene (PS), and polycarbonate (PC) are used. The arrangement of the transparent housing structure enables the user to constantly observe the position of the ultrasonic transducer 220 and the state of the body part to be scanned in the process of operating the imaging device, thereby enabling the user to easily make operation decisions. In contrast, the prior art does not include the above detachable housing structure, and transparent materials cannot be selected since the frame 301 as a whole needs to be made of a material with a higher mechanical strength (for example, metal); therefore, it is difficult to achieve the clear observation effect accomplished in the present disclosure.

Referring to FIG. 6, a schematic diagram of a connection relationship between the frame 301 and the ultrasonic transducer 220 is shown according to some embodiments of the present disclosure. The drawing omits the structure of a housing that accommodates the driving device 230 in the frame 301, and the structure including a controller and a circuit board arranged inside the frame 301, so as to clearly illustrate the specific structure of the driving device 230. The driving device 230 is partially accommodated in the frame 301 (the specific housing is not shown), and is connected to the ultrasonic transducer 220 so as to be able to drive the ultrasonic transducer 220 to move. The driving device 230 may include a motor 601 and a screw rod 602. The screw rod 602 is arranged in the frame 301, for example, two ends thereof may be fixedly connected to two side walls of the frame 301. The motor 601 is connected to the ultrasonic transducer 220. For example, the motor 601 may be fixedly connected to one end of the ultrasonic transducer 220 by means of a screw structure. The motor 601 is movably connected to the screw rod 602 so as to drive the ultrasonic transducer 220 to move.

In the configuration shown in FIG. 6, the motor 601 drives the ultrasonic transducer 220 to perform reciprocating movement in the direction of the screw rod by means of rotation of an internal output shaft in different directions (for example, a clockwise or counterclockwise direction) or by using the output shaft to drive a gear to movably connect to the screw rod 602, thereby automatically performing ultrasonic scanning. It should be noted that other motion modes are also allowed. For example, the ultrasonic transducer 220 is driven to rotate around some center of a circle. Although the internal structure of the motor 601 is not directly shown in FIG. 6, those skilled in the art know that all configurations that establish the movable connection between the motor 601 and the screw rod 602 are allowed.

In order to further improve the stability of the movement of the ultrasonic transducer 220, an additional mechanical structure may be further arranged in an exemplary embodiment. For example, the driving device 230 may further include a guide rail and a sliding block. As shown in FIG. 6, the guide rail 604 may be fixedly arranged in the frame 301 in any manner, for example, fixed by screws. For example, the guide rail 604 may be fixed to the two side walls of the frame 301 in a manner of being substantially parallel to the screw rod 602. Correspondingly, the sliding block 603 may be fixedly connected to the motor 601. The fixed connection may be established when the sliding block and the motor are integrally formed, or welded together or detachably connected in any other manner. The sliding block 603 and the guide rail 604 are configured to be slidably connected, so as to perform a guiding function when the ultrasonic transducer 220 moves. Such an arrangement ensures that the ultrasonic transducer 220 moves more stably when performing automatic scanning, and is not easily impacted by other conditions that affect the quality of ultrasonic imaging, such as side-to-side oscillations or up-and-down vibrations.

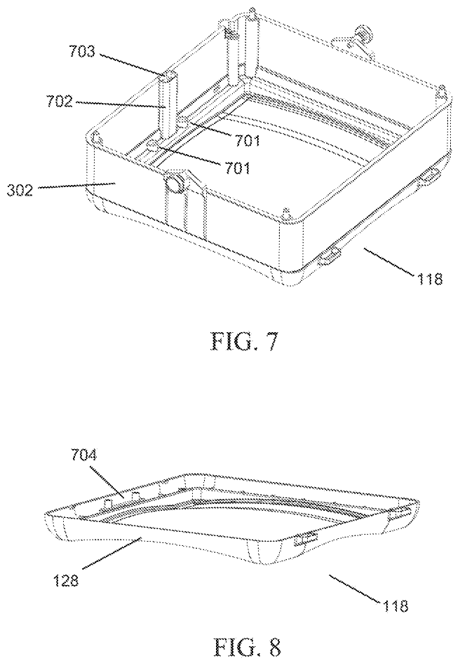

The lower housing 302 and the film assembly 118 may be detachably connected in a variety of manners, for example, detachably connected by means of a magnetic force. Refer to FIG. 7 and FIG. 8. FIG. 7 shows a perspective view of the lower housing 302 and the film assembly 118 in an assembled state according to some embodiments of the present disclosure. FIG. 8 shows a perspective view of the film assembly at one angle according to some embodiments of the present disclosure. In an exemplary embodiment, a lower open end of the lower housing 302 may be provided with a first magnetic material 701, and the outer frame 128 of the film assembly 118 is provided with a second magnetic material 704 correspondingly. The first magnetic material 701 and the second magnetic material 704 attract each other through a magnetic force. As a result, the film assembly 118 is connected to the lower open end of the lower housing 302 as shown in FIG. 7. In an exemplary embodiment, the first and second magnetic materials may be permanent magnet materials, such as magnets, and surfaces of the two magnetic materials facing each other have opposite magnetic poles, so that they can attract each other. Alternatively, one of the first and second magnetic materials is a permanent magnet material, and the other is a ferromagnetic material such as iron, cobalt, nickel, or an alloy thereof. Examples are not exhaustively enumerated herein. In an exemplary embodiment, as shown in FIG. 7 and FIG. 8, the first magnetic material 701 may include two recesses (not shown in FIG. 7), and correspondingly, the second magnetic material may include two protrusions. The recesses and the protrusions match each other. On the one hand, attraction between the film assembly 118 and the lower housing 302 can be enhanced; on the other hand, the two magnetic materials can also limit the position of the film assembly to avoid sliding of the film assembly relative to the lower housing. It should be noted that FIG. 7 and FIG. 8 only show an arrangement example of a set of first magnetic material 701 and second magnetic material 704, but the quantities, shapes, and positions of the two magnetic materials can be arbitrarily determined as long as the attraction between the two magnetic materials is greater than the weight of the film assembly 118.

The detachable design of the film assembly 118 enables the user to easily detach and replace the film assembly 118 with a new one after the scanning is completed. However, if a scan is performed directly without installation of a new film assembly 118, it is very disadvantageous for the personal safety of a subject to be scanned and for the protection of the ultrasonic transducer 220. In an exemplary embodiment, a detection circuit is provided for detecting whether the film assembly 118 is reliably installed at the lower open end of the lower housing 302. Refer to FIG. 4, FIG. 7, and FIG. 8. The detection circuit may include a magnetic induction switch 706 and an electrical connection assembly. The magnetic induction switch 706 is arranged at the lower open end 322 of the lower housing 302, and the electrical connection assembly electrically connects the magnetic induction switch 706 to a controller. The controller may be arranged in the scanning assembly 108 (not shown), or may be arranged in the scanning processor. In this way, when the film assembly 118 is connected to the lower open end 322 of the lower housing 302, the second magnetic material 704 will be close enough to the magnetic induction switch 706. At this time, the magnetic induction switch 706 can sense a magnetic field from the second magnetic material 704, such that a path is formed, and a signal is sent to the controller by means of the electrical connection assembly. At this time, the controller senses that the film assembly 118 is installed at the lower open end 322 of the lower housing 302, such that a control signal can be sent to allow the motor 601 and the ultrasonic transducer 220 to operate normally. Conversely, if no electrical signal is received from the detection circuit, the controller is configured to send a control signal to prohibit the motor 601 and the ultrasonic transducer 220 from operating. In an exemplary embodiment, the detection result of the above detection circuit is further configured to be displayed on the display 110 shown in FIG. 2. Such an arrangement can prevent an accident from occurring when a user forgets to install a new film assembly after removing the film assembly 118, and when the film assembly 118 falls off in the ultrasonic scanning process. It should be noted that the magnetic induction switch is configured to be at a position unaffected by a magnetic field of the first magnetic material 701, so as to avoid signal generation when the film assembly 118 is not installed. The type of the magnetic induction switch 706 may be any type in the art. In an exemplary embodiment, the magnetic induction switch 706 may be a reed switch such as a dry reed switch. In other embodiments, the magnetic induction switch 706 may be a Hall switch. The present disclosure has made a detailed description of the detection circuit. On this basis, the type and configuration of the magnetic induction switch 706 may be arbitrary.

Some exemplary embodiments of the specific arrangement of the electrical connection assembly will be provided below. Continue to refer to FIG. 4, FIG. 7, and FIG. 8. In an exemplary embodiment, the electrical connection assembly may include a conductive element 702, a first electrical contact 703, and a second electrical contact 705. In an exemplary embodiment, the conductive element 702 may include a wire arranged at a side portion of the lower housing 302. One end of the wire is connected to the magnetic induction switch 706, and the other end is connected to the first electrical contact 703. The first electrical contact 703 may be arranged at a portion of the upper open end 312 of the lower housing 302 connected to the frame 301. Correspondingly, the second electrical contact 705 may be arranged at the bottom of the frame 301 and correspond to the first electrical contact. In this way, when the lower housing 302 and the frame 301 are assembled, the first and second electrical contacts are electrically connected. The second electrical contact 705 may be further electrically connected to the controller. The electrical connection manner will not be described here. It should be noted that the above installation manner of the electrical connection assembly is not the only one, and there are other potential manners, which will not be described here.

The purpose of providing the above specific embodiments is to facilitate understanding of the content disclosed in the present disclosure more thoroughly and comprehensively, but the present disclosure is not limited to these specific embodiments. Those skilled in the art should understand that various modifications, equivalent replacements, and changes can also be made to the present disclosure and should be included in the scope of protection of the present disclosure as long as these changes do not depart from the spirit of the present disclosure.

What is claimed is:

1. A scanning assembly for an ultrasonic imaging device, comprising:
an ultrasonic transducer configured to send/receive an ultrasonic signal;
a frame connected to an ultrasonic imaging device body;
a lower housing comprising a hollow cavity having an upper open end and a lower open end, wherein the upper open end is detachably connected to the bottom of the frame, and the ultrasonic transducer is at least partially accommodated in the hollow cavity; and
a film assembly detachably connected to the lower open end.

2. The scanning assembly according to claim 1, wherein the upper open end of the lower housing and the bottom of the frame are respectively provided with a plurality of guideposts and a plurality of guide holes corresponding to one another.

3. The scanning assembly according to claim 1, wherein the upper open end of the lower housing and the bottom of the frame are respectively provided with a locking element and a mating element corresponding to the locking element.

4. The scanning assembly according to claim 3, wherein the locking element comprises a pin, and the mating element comprises a pin hole.

5. The scanning assembly according to claim 3, wherein the locking element comprises a hook, and the mating element comprises a recess.

6. The scanning assembly according to claim 1, wherein the lower housing is made of a transparent material.

7. The scanning assembly according to claim 1, further comprising an upper housing detachably connected to the top of the frame.

8. The scanning assembly according to claim 1, wherein the upper housing is made of a transparent material.

9. The scanning assembly according to claim 1, further comprising an illumination device arranged inside the scanning assembly and configured to illuminate tissue to be imaged in an ultrasonic imaging process.

10. The scanning assembly according to claim 1, further comprising a driving device at least partially arranged in the frame, wherein the driving device is connected to the ultrasonic transducer to drive the ultrasonic transducer to move.

11. The scanning assembly according to claim 10, wherein the driving device comprises a motor and a screw rod, the screw rod is arranged in the frame, and the motor is fixedly connected to the ultrasonic transducer and movably connected to the screw rod to drive the ultrasonic transducer to move.

12. The scanning assembly according to claim 11, wherein the driving device further comprises a guide rail and a sliding block, the guide rail is fixedly arranged in the frame, the sliding block is fixedly connected to the motor, and the guide rail is in slide connection with the sliding block and is configured to perform a guiding function when the ultrasonic transducer moves.

13. The scanning assembly according to claim 1, wherein the film assembly comprises an outer frame and a film, the film is fixedly arranged in the outer frame, the outer frame is detachably connected to the lower open end of the lower housing, and in an ultrasonic imaging process of the ultrasonic imaging device, one surface of the film is at least partially in contact with the ultrasonic transducer, and the other surface of the film is at least partially in contact with the tissue to be scanned.

14. The scanning assembly according to claim 13, wherein the lower open end of the lower housing is provided with a first magnetic material, the outer frame is provided with a second magnetic material correspondingly, and the first magnetic material and the second magnetic materials attract each other through a magnetic force, such that the film assembly is connected to the lower open end of the lower housing.

15. The scanning assembly according to claim 14, further comprising a detection circuit, wherein the detection circuit comprises a magnetic induction switch arranged at the lower open end of the lower housing and an electrical connection assembly connecting the magnetic induction switch to a controller, and when the film assembly is connected to the lower open end of the lower housing, the magnetic induction switch senses a magnetic field from the second magnetic material, and sends a signal to the controller through the electrical connection assembly.

16. An ultrasonic imaging device, comprising:
an ultrasonic imaging device body comprising a main device, a display, and an adjustable arm; and
the scanning assembly according to any one of the above claims, wherein the scanning assembly is connected to one end of the adjustable arm.
